(19) United States
(12) Patent Application Publication
(10) Pub. No.: US 2026/0226536 A1
(43) Pub. Date: Aug. 6, 2026

(54) MARKERS OF DISEASE

(71) Applicant: NUCLEIX LTD., Rehovot (IL)

(72) Inventors: Danny FRUMKIN, Rehovot (IL); Revital KNIRSH, Rosh HaAyin (IL); Orna SAVIN, Herzliya (IL); Adam WASSERSTROM, Ness Ziona (IL); Nimrod AXELRAD, Rehovot (IL)

(73) Assignee: NUCLEIX LTD., Rehovot (IL)

(21) Appl. No.: 19/150,485

(22) PCT Filed: Jan. 25, 2024
(86) PCT No.: PCT/IL2024/050099

§ 371 Date: Jul. 23, 2025

(30) Foreign Application Priority Data
Jan. 26, 2023 (IL) 300230

(51) Int. Cl. C12Q 1/6869; C12Q 1/6806; C12Q 1/683; G16B 35/20; G16H 10/40; G16H 10/60; G16H 50/30; G16H 50/70

(52) U.S. Cl. CPC C12Q 1/6869; C12Q 1/6806; C12Q 1/683; G16B 35/20; G16H 10/40; G16H 10/60; G16H 50/30; G16H 50/70; C12Q 2600/118; C12Q 2600/154

(57) ABSTRACT

The invention relates to methods for identifying and using markers of disease, in particular based on methylation levels in cfDNA CpG loci.

### FIELD OF THE INVENTION

The invention relates to methods for identifying and using markers of disease, in particular based on methylation levels of cfDNA CpG loci.

### BACKGROUND OF THE INVENTION

Markers are increasingly relied upon for assessment of disease. Markers can be identified using databases of the molecular characteristics of disease. The Cancer Genome Atlas (TCGA), for example, aimed to generate and interpret molecular characteristics at the DNA, RNA, protein and epigenetic levels for hundreds of clinical tumors representing various tumor types and subtypes—Cancer Genome Atlas Research Network et al., Nat Genet. 2013; 45(10): 1113-1120.

TCGA can be analysed to identify markers of cancer that are present throughout populations, and that are specific to particular tumor types. However, TCGA focuses on genome mutations that are detectable in diseased tissue. Furthermore, because TCGA is based on bisulfite sequencing (which has a high noise level), TCGA markers cannot easily be detected above background at low levels. In plasma, the majority of acellular DNA is from healthy tissue, meaning that markers of diseased tissue are present in low levels—making the detection of rare TCGA mutations in plasma unfeasible. There remains a need for identification of new markers that allow for more specific and/or sensitive assessment of diseases using non-invasive techniques.

Alterations in DNA methylation levels are one of the earliest, most common characteristics of many diseases, and may be measured in plasma, making differential methylation an ideal marker type. Methylation profiling of cell-free DNA (cfDNA) has significant potential to expand the use of liquid biopsies for assessment of disease. For instance, liquid biopsy and circulating tumor DNA (ctDNA) are established plasma surrogates of tumor tissue, but their use in early detection remains challenging due to the high noise level of current techniques. In addition, due to the heterogeneity of many diseases (such as cancer), no single methylation marker has allowed assessment of disease, for example definitive diagnosis of disease or determination of disease classification. A panel of methylation markers is desirable for specificity and/or sensitivity. The total number of markers used can be limited to make testing practicable and affordable.

There is therefore a need for methods of selecting a panel of markers of a disease for specific and/or sensitive assessment of diseases such as cancer. Preparing a database comprising cfDNA CpG loci and methylation levels, provides a platform to interrogate diseases and select panels of markers.

### SUMMARY OF THE INVENTION

The invention provides a method of preparing a database comprising cfDNA CpG loci, the method comprising:
(a) Providing DNA extracted from:
   (i) a sample of disease tissue from one or more first subjects with a disease;
   (ii) a sample comprising cfDNA from the one or more first subjects; and
   (iii) a sample comprising cfDNA from one or more second subjects without the disease,
(b) Digesting at least a portion of the DNA with one or more methylation-sensitive restriction enzymes (MSREs) and/or one or more methylation-dependent restriction enzymes (MDREs), producing digested DNA;
(c) Sequencing the digested DNA with high-throughput sequencing, thereby identifying cfDNA CpG loci;
(d) Determining methylation levels of the cfDNA CpG loci in the samples of (i), the samples of (ii), and the samples of (iii); and
(e) Preparing the database by recording:
   the cfDNA CpG loci identified by the sequencing in (c);
   the methylation levels determined in (d) for the cfDNA CpG loci;
   a sample identifier; and
   a subject identifier.

The invention further provides a method of preparing a database comprising cfDNA CpG loci, the method comprising:
(a) Providing DNA extracted from:
   (i) a sample of disease tissue from a plurality of first subjects with a disease; and
   (ii) a sample comprising cfDNA from the plurality of first subjects,
(b) Digesting at least a portion of the DNA with one or more methylation-sensitive restriction enzymes (MSREs) and/or one or more methylation-dependent restriction enzymes (MDREs), producing digested DNA;
(c) Sequencing the digested DNA with high-throughput sequencing, thereby identifying cfDNA CpG loci;
(d) Determining methylation levels of the cfDNA CpG loci in the samples of (i) and the samples of (ii); and
(e) Preparing the database by recording:
   the cfDNA CpG loci identified by the sequencing in (c);
   the methylation levels determined in (d) for the cfDNA CpG loci;
   a sample identifier; and
   a subject identifier,
wherein the plurality of first subjects comprises one or more primary first subjects with a disease classification, and one or more other first subjects without the disease classification.

The invention further provides a method for identifying at least one differentially methylated marker of disease, the method comprising:
(A) Providing a database comprising cfDNA CpG loci prepared according to a method of the invention;
(B) Querying the database for cfDNA CpG loci having different methylation levels between the one or more first subjects and the one or more second subjects;
(C) Ranking the cfDNA CpG loci according to statistical significance of the difference in methylation levels; and
(D) Identifying as markers, cfDNA CpG loci having a statistically significant difference in methylation levels.

The invention further provides a method for identifying at least one differentially methylated marker of disease, the method comprising:
(A) Providing a database comprising cfDNA CpG loci prepared according to a method of the invention;
(B) Querying the database for cfDNA CpG loci having different methylation levels between the one or more primary first subjects and the one or more other first subjects;
(C) Ranking the cfDNA CpG loci according to statistical significance of the difference in methylation levels; and
(D) Identifying as markers, cfDNA CpG loci having a statistically significant difference in methylation levels.

The invention further provides a method of selecting a panel of differentially methylated markers of disease, the method comprising:
(I) Providing differentially methylated markers of disease identified according to a method of the invention; and
(II) Selecting at least about 10 of the differentially methylated markers, in order to obtain an area under a ROC curve (AUC) of at least about 0.8 when the markers are used to assess test subjects.

The invention further provides a panel of markers selected according to a method of the invention.

The invention further provides a kit for assessing a disease, comprising means for detecting a panel of markers of the invention in a sample comprising cfDNA.

The invention further provides an in vitro method for assessing disease, the method comprising detecting a panel of markers of the invention.

### BRIEF DESCRIPTION OF THE DRAWINGS

FIG. 1 provides a schematic of an embodiment of the invention, showing four sample types from subjects with a disease such as cancer. The samples can be compared in several ways, shown collectively in panel A. These include comparing methylation levels in samples from a plurality of subjects (panel B); comparing methylation levels and non-methylation analytes in plasma samples of a plurality of subjects in a ‘multiomics’ approach (panel C); and comparing methylation levels between subjects with different diagnoses or disease characteristics (panel D).

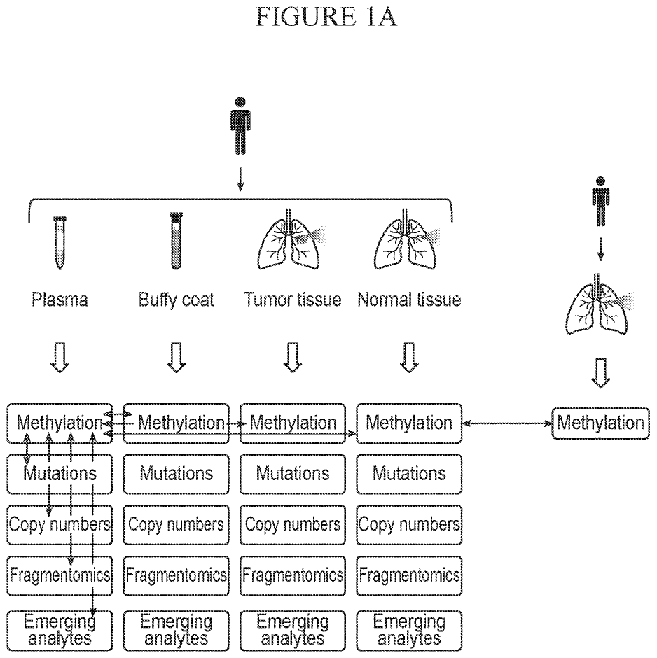

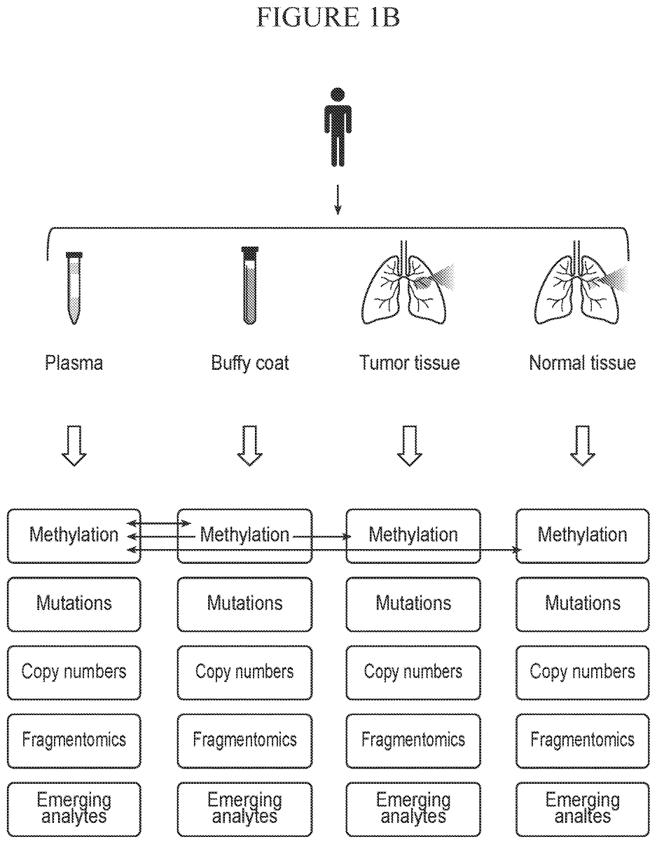

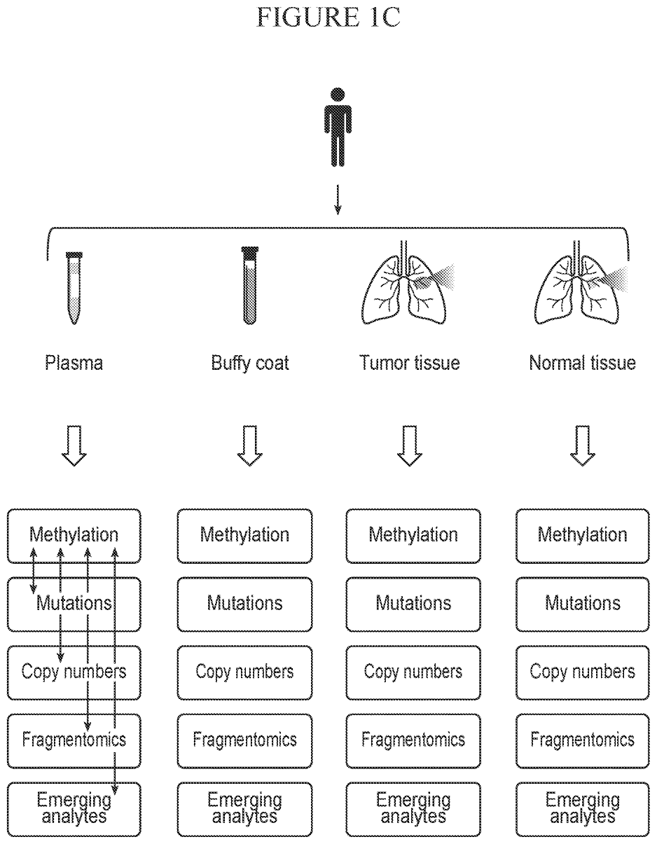

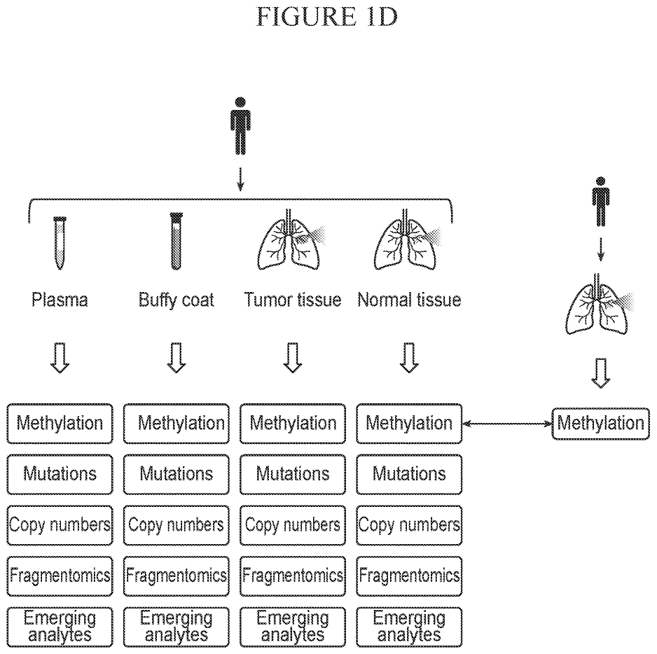

### DETAILED DESCRIPTION OF THE INVENTION

### Definitions

The practice of the present invention will employ, unless otherwise indicated, conventional methods of chemistry, biochemistry, and molecular biology, within the skill of the art. Such techniques are explained fully in the literature. See e.g. Methods In Enzymology (Academic Press, Inc.), Green & Sambrook (2012) Molecular Cloning: A Laboratory Manual, 4th edition (Cold Spring Harbor Press), Ausubel et al. (eds) Short protocols in molecular biology, 5th edition (Current Protocols), Molecular Biology Techniques: An Intensive Laboratory Course, (Ream & Field, eds., 1998, Academic Press), Wilson and Walker's Principles and Techniques of Biochemistry and Molecular Biology (Hodmann & Clokie, 2018), Basic Molecular Biology & Techniques—Recent Advances: Molecular Biology & Its Technique (Singh et al., 2021), etc.

The various steps of methods may be carried out at the same or different times, in the same or different geographical locations, e.g. countries, and by the same or different people or entities. Various features of the methods, such as samples, are denoted with Roman numerals, such as ‘(i)’, ‘(ii)’, ‘(iii)’, etc. Features listed in a given embodiment need not have consecutive numerals in a complete numeric set-for example, some methods may use samples (i), (ii), (v) and (vi), and such methods need not also comprise samples denoted ‘(iii)’ and ‘(iv)’. Numerals are indicative of specific features but not necessarily the order of features, and thus non-consecutive numerals are acceptable.

Use of the term “about” when referring to a quantity is optional, and means that the quantity may be varied according to what the skilled person would recognise as the structure or function associated with the quantity. For example, sequencing to an average depth of “about 600×” indicates that sequencing to an average depth of less than or more than 600× is acceptable provided that the desired outcome (e.g. accurate sequencing) may be achieved. In some embodiments, “about” indicates variation in a quantity of +/−10% of the quantity, e.g. “about 600×” may indicate “between 540× and 660×”.

“Between” (and variations such as “x to y”, “x-y”), with reference to two values, includes those two values e.g. the range “between” 10 mg and 20 mg encompasses inter alia 10, 15, and 20 mg.

“Cell-free DNA” (“cfDNA”) refers to fragmented genomic DNA which is found in vivo in an animal within a bodily fluid rather than within an intact cell. cfDNA is highly fragmented compared to intact genomic DNA (e.g. see Alcaide et al. (2020) Scientific Reports 10, article 12564), and in general circulates as fragments between 120-220 bp long, with a peak around 168 bp (in humans). cfDNA is present in many bodily fluids, including but not limited to blood and urine. cfDNA may enter the blood following apoptosis or necrosis. cfDNA may be unbound, or bound to histones.

“Circulating tumor DNA” (“ctDNA”) refers to CfDNA originating from a tumor cell.

The term “comprising” encompasses “including” as well as “consisting of”, e.g. a composition “comprising” X may consist exclusively of X or may include something additional (e.g. X+Y).

“Depth” refers to the number of times a particular locus in a reference genome is spanned, or is terminated at, by sequence reads produced in high-throughput sequencing and aligned to the genome. Hence, depth can be assessed from aligned sequence reads which terminate at or span a locus. For a particular locus, a depth of “600×” indicates 600 assessments of the methylation status of the locus according to such alignments. In the context of sequencing depth, the term “average” refers to the arithmetic mean. Thus, the “average depth” can be calculated by dividing the sum of nucleotides in sequence reads which map to the human genome by the length of the (haploid) human genome.

High depth is useful for determining whether differences in the sequence reads with respect to a reference sequence reflect the underlying sequence of the sample DNA (signal) or are due to errors during sequencing (noise). High depth therefore aids in drawing meaningful conclusions from sequencing data, particularly regarding the presence of rare signals, such as those that result from tumor DNA. Methylated cfDNA molecules from a tumor may be present in plasma at amounts in the order of ≤1% of the total cfDNA. Bisulfite sequencing does not provide sufficient depth across a large enough number of loci to reliably detect these rare signals. In contrast, the methods of the invention have very high average depths, so allowing the detection of these rare signals.

Any particular CpG site can give rise to different types of sequencing read depending on whether it is methylated or unmethylated. For instance, if a MSRE is used and a particular CpG is methylated then the cfDNA molecule will not be cleaved and the intact recognition site will be seen in a sequencing read (and a sequence alignment will span the recognition site completely). Conversely, if the CpG is unmethylated then the molecule will be cleaved and reads of the molecule will start or end with the sequence resulting from the cleavage (but will still align to the reference genome at the relevant locus). As some library preparation methods can result in depletion of small fragments, which are then not sequenced (e.g. in CpG islands, where a starting cfDNA molecule is cleaved by a MSRE at more than one unmethylated site, thus providing 3 or more restriction fragments, some of which are very small), the observed number of unmethylated CpG sites may be lower than the true value in the original sample. This distortion can be somewhat addressed by using the larger of the number of reads whose 3′ ends map to a site and the number of reads whose 5′ ends map to a site (or to use the mean).

One way of expressing coverage of a particular locus or CpG site is referred to as “Hitspan100”, which refers to the number of sequence reads which span a certain CpG position with at least 50 nucleotides both upstream and downstream. For example, a Hitspan 100 of 90 at a specific CpG site means that there are 90 sequence reads which span this site with at least 50 nucleotides both upstream and downstream. Comparing the Hitspan 100 of a digested sample with that of an undigested sample can also be used to address the above distortion caused by depletion of small fragments.

“Digestion” (and also “digesting”, etc.) refers to the mixing of active restriction enzymes with DNA in conditions under which DNA cleavage can occur. If there are no recognition sites for the restriction enzymes in question (e.g. because the restriction enzymes are MSREs and all of the recognition sites are fully methylated) then a step of “digestion” still takes place even though DNA cleavage does not occur.

“Extraction” or “purification” refers to the removal of other substances from a desired substance. The desired substance need not be isolated completely from other substances (i.e. 100% purity need not be reached, though it may be). Rather, extraction involves removal an adequate amount of other substances, e.g. to prepare the desired substance for further processing. In the context of DNA, extraction may comprise removal of tissue, enzymes, other proteins, and/or lipids, to enable the DNA to be digested and sequenced, or otherwise used with methods of the invention.

“Locus” refers to a specific location within the genome, and may include a single position (a single nucleotide at a defined position in the genome), a dinucleotide (such as a CpG site), or a stretch of nucleotides starting and ending at defined positions in the genome. The specific position(s) may be identified by the molecular location in a reference genome (such as the Hg38 assembly of the human genome), namely, by the chromosome and the numbers of the starting and ending base pairs on the chromosome. In some embodiments, a locus comprises at least one CpG site. A “cfDNA CpG locus” refers to a locus comprising a CpG site (or the cytosine residue of a CpG site), detectable in cfDNA. “cfDNA non-CpG locus” refers to a locus not comprising a CpG site (or the cytosine residue of a CpG site), detectable in cfDNA.

“Matched” refers to subjects that share one or more characteristics. Subjects may be matched to reduce sources of variability between their samples, and to aid in identifying clinically relevant differences.

Samples used in methods of preparing the database may be autologous (derived from the same subject). For example, a set of sample types (such as a sample of disease tissue and a sample comprising cfDNA; or a sample of disease tissue, a sample of healthy tissue, a sample comprising cfDNA, and a sample comprising nucleated blood cells) may be provided from the same subject. Samples may be derived from the same organ type or same tissue type. For example, a sample of disease tissue and a sample of healthy tissue from a subject may be derived from the same organ type or tissue type. For example, if a subject with a disease has one diseased lung and one healthy lung, the diseased tissue and healthy tissue may both be provided from different parts of the diseased lung, or the diseased tissue may be provided from the diseased lung and the healthy tissue provided from the other, healthy, lung.

However, samples need not always be autologous. Samples from different subjects may be used provided that they are matched—for instance, samples from twins or immediate blood relatives may be used. Samples may also be obtained from subjects who are not blood relatives, but who are nevertheless matched according to specific criteria. Subjects used in some embodiments are matched by having the same disease (as first subjects) or by not having the disease (as second subjects). Subjects used in some embodiments are matched by having the same disease and the same disease classification (as primary first subjects) or by having the same disease but not having the disease classification (as other first subjects). Subjects and subject groupings are discussed below.

Methods of preparing the database may involve matching subjects using further criteria, in addition to the matching by disease or disease classification described above. For example, methods of the preparing the database may involve subjects that are matched according to at least one demographic criterion, such as age, race, sex, ethnicity; and/or at least one non-demographic criterion, such as health status, medical history, disability status, body weight, BMI, smoking status, chronic obstructive pulmonary disease (COPD) status, tumor presence, tumor size, cancer stage, cancer metastasis, and psychiatric diagnosis.

Matched samples may be provided from the same organ type or tissue type from the different subjects—for example, samples of healthy tissue may be provided from the lungs of different subjects.

“Methylation-sensitive restriction enzymes” or “MSREs” are restriction enzymes that cleave target DNA only if a CpG within the recognition site is unmethylated, and methylation inhibits the cleavage.

“Methylation-dependent restriction enzymes” or “MDREs” are restriction enzymes that cleave target DNA only if a CpG within the recognition site is methylated.

MSREs and MDREs are readily available from well-known commercial suppliers, such as ThermoFisher, New England Biolabs, Promega, etc.

A “pack year” is a unit of smoking equivalent to an average of 1 pack of cigarettes (such as 20 cigarettes) per day for 1 year. For example, a person could have a 20 pack year history by smoking 1 pack a day for 20 years, or 2 packs a day for 10 years.

A “receiver operator characteristic” curve, or “ROC” curve, is a plot of sensitivity versus (1—specificity) for a diagnostic test. The area under a ROC curve (AUC) is an indicator of the overall diagnostic accuracy of a test. See also Makdrekar (J Thorac Oncol. 2010; 5:1315-1316).

### Methylation and CpG Sites

Methods of preparing a database comprising cfDNA CpG loci as disclosed herein involve determining methylation levels. In eukaryotic DNA, 5-methyl modifications of cytosine occur in the context of CG dinucleotide sequences (commonly denoted as ‘CpG’ dinucleotides or ‘CpG sites’). CpG sites are not randomly distributed throughout eukaryotic genomes, and are frequently found in clusters known as ‘CpG islands’. These islands have been formally defined as regions which are at least 200 bp long, having 50% or more GC content, and where the observed-to-expected CpG ratio is greater than 60% (i.e. where the number of CpG sites multiplied by the length of the sequence, divided by the number of C multiplied by the number of G, is greater than 0.6). CpG islands are often found near the start of a gene in mammalian genomes, and about 70% of promoters near transcription start sites in the human genome contain a CpG island. The methylation status of multiple sites within a single CpG island tends to be the same (referred to as co-methylation). Methylation of multiple CpG sites within a promoter's CpG island is generally associated with stable silencing of gene expression from that promoter.

The human genome sequence contains around 28 million CpG sites (per haploid genome), with around 30,000 CpG islands. In any particular nucleated cell some CpG sites will be methylated and others will not. Patterns of methylation can differ between different cells and tissues within a subject, such that a specific CpG can be methylated in one cell or tissue but unmethylated in a different cell or tissue within the same subject.

It is known that tumor cells can display different methylation patterns compared to non-tumor cells (or compared to other types of tumor cell). Some CpG sites can become hypermethylated in tumors, while others can become hypomethylated. cfDNA CpG loci that exhibit notably different methylation levels between samples may be used as markers of disease.

### Samples, Subjects, and Matching

Methods of preparing the database use DNA extracted from samples, including cfDNA and genomic DNA. The samples include samples of tissue, such as samples of disease tissue and/or samples of healthy tissue, which comprise genomic DNA. The samples also include samples of plasma, which comprise cfDNA, for example ctDNA. Samples of nucleated blood cells also comprise cfDNA.

cfDNA is present in many bodily fluid samples, including but not limited to blood and urine, and methods of the invention can use any suitable source of cfDNA e.g. a sample of whole blood (such as a sample of venous blood) or a sample of urine. Ideally cfDNA is isolated from a sample of whole blood, and the sample of whole blood may be treated to yield plasma (i.e. the liquid remaining after a sample of whole blood is subjected to a separation process to remove the blood cells, typically involving centrifugation) or serum (i.e. blood plasma without clotting factors such as fibrinogen). Preferably, samples comprising cfDNA are samples of plasma. Thus, markers identified according to methods of the invention can be used as part of so-called liquid biopsy testing.

It is preferred that samples of buffy coat and plasma are obtained from the same sample of whole blood (i.e. a sample of whole blood may be provided and separated into a sample of plasma and a sample of buffy coat, such that samples of buffy coat and plasma are obtained together). However, the samples of plasma and buffy coat can be obtained from different samples of whole blood (i.e. samples of plasma and buffy coat can be obtained separately). In some embodiments, samples of whole blood have a volume of 10 ml or less.

Methods of preparing the database may begin with DNA which has already been extracted from samples. Alternatively, the methods may include an upstream step of extracting the DNA from samples. Similarly, the methods may include an upstream step of obtaining samples as well as a step of extracting DNA from the samples.

Samples are provided from: one or more first subjects with a disease and one or more second subjects without a disease; or, from a plurality of first subjects, comprising primary first subjects with a disease classification and other first subjects without the disease classification. These two aspects are discussed below.

The samples from the one or more first subjects, or from the plurality of first subjects, comprise (i) a sample of disease tissue and (ii) a sample comprising cfDNA (such as a sample of plasma).

In some embodiments, the samples from the one or more first subjects, or from the plurality of first subjects, also comprise (v) a sample of healthy tissue and/or (vi) a sample comprising nucleated blood cells (such as a sample of buffy coat). Samples (v) and (vi) are useful, for example, for identifying cfDNA CpG loci with different methylation levels in disease tissue compared to healthy tissue. In some embodiments, the samples of disease tissue and healthy tissue are obtained from the same organ type and/or from the same organ. For example, if a subject with a disease has one diseased lung and one healthy lung, the sample of diseased tissue and the sample of healthy tissue may both be provided from the diseased lung, or the sample of diseased tissue may be provided from the diseased lung and the sample of healthy tissue provided from the other, healthy, lung. In some embodiments, the disease tissue and healthy tissue comprise the same tissue type. In some embodiments, the disease tissue and healthy tissue comprise the same cell type.

In embodiments with a plurality of first subjects, the plurality of first subjects comprises subjects with the same disease, but with different disease classifications. For example, the plurality of first subjects may comprise one or more primary first subjects with one disease classification (such as one stage of a disease, one subtype of a disease, one type of diseased tissue, one disease severity level, one prognosis, or one degree of amenability to treatment), and one or more other first subjects not comprising the disease classification (such as having a different stage of a disease, a different subtype of disease, a different type of disease tissue, a different prognosis, a different severity, or a different amenability to treatment). Where the one or more primary first subjects and one or more other first subjects differ in the stage of disease, the one or more primary first subjects may have a different cancer stage to the one or more other first subjects, such as stage I cancer in the one or more primary first subjects and stage II cancer in the one or more other first subjects. Where the one or more primary first subjects and the one or more other first subjects differ in the type of diseased tissue, the one or more primary first subjects may have cfDNA CpG loci originating from a disease in a different cell type, tissue and/or organ compared to the one or more other first subjects, such as cancers in different tissues.

As the one or more primary first subjects have the same disease classification, they are matched according to this characteristic. The one or more primary first subjects may be further matched by at least one demographic criterion and/or at least one non-demographic criterion.

As the one or more other first subjects do not have the disease classification, they are matched according to this characteristic. The one or more other first subjects may be further matched by at least one demographic criterion and/or at least one non-demographic criterion.

The one or more primary first subjects may be matched with the one or more other first subjects by at least one demographic criterion and/or at least one non-demographic criterion. Thus, in some embodiments, the one or more primary first subjects are further matched amongst themselves by at least one demographic criterion and/or at least one non-demographic criterion, the one or more other first subjects are further matched amongst themselves by at least one demographic criterion and/or at least one non-demographic criterion, and the one or more primary first subjects are matched with the one or more other first subjects by at least one demographic criterion and/or at least one non-demographic criterion. In some embodiments, the same demographic criterion and/or non-demographic criterion is used to further match the one or more primary first subjects amongst themselves, to further match the one or more other first subjects amongst themselves, and to match the one or more primary first subjects with the one or more other first subjects.

Methods of preparing the database may be performed with samples from any number of subjects that is adequate to provide the statistical power required in the technical field, for example, to assess subjects as having a disease or as having a disease classification, to a clinically accepted standard.

The number of primary first subjects and the number of other first subjects may be the same, or may be different.

In some embodiments, the plurality of first subjects comprises at least about 10, at least about 20, at least about 30, at least about 40, at least about 50, at least about 60, at least about 70, at least about 80, at least about 90, or at least about 100 subjects. Preferably, the plurality of first subjects comprises at least about 80 subjects.

In some embodiments, the one or more primary first subjects comprises at least about 5, at least about 10, at least about 15, at least about 20, at least about 25, at least about 30, at least about 35, at least about 40, at least about 45, at least about 50, at least about 60, at least about 70, at least about 80, or at least about 90 subjects. Preferably, the one or more primary first subjects comprises at least about 40 subjects.

In some embodiments, the one or more other first subjects comprises at least about 5, at least about 10, at least about 15, at least about 20, at least about 25, at least about 30, at least about 35, at least about 40, at least about 45, at least about 50, at least about 60, at least about 70, at least about 80, or at least about 90 subjects. Preferably, the one or more other first subjects comprises at least about 40 subjects.

In some embodiments, the one or more primary first subjects comprises at least about 40 subjects and the one or more other first subjects comprises at least about 40 subjects.

In preferred embodiments, the plurality of first subjects is human.

Methods of preparing the database do not require healthy (control) subjects-for example, healthy subjects are not required in embodiments featuring a plurality of first subjects with different disease classifications as above (although one or more healthy subjects may be beneficial in those embodiments).

However, some embodiments do feature healthy subjects without the disease. Thus, in another aspect, methods of preparing the database may involve one or more first subjects with a disease, and one or more second subjects without the disease. Thus, in some embodiments, samples are provided from one or more first subjects with a disease and from one or more second subjects without the disease.

As for the plurality of first subjects, the samples from the one or more first subjects comprise (i) a sample of disease tissue and (ii) a sample comprising cfDNA (such as a sample of plasma). In some embodiments, the samples from the one or more first subjects further comprise (v) a sample of healthy tissue and/or (vi) a sample comprising nucleated blood cells (such as a sample of buffy coat).

The samples from the one or more second subjects comprise (iii) a sample comprising cfDNA (such as a sample of plasma). In some embodiments, the samples from the one or more second subjects further comprise (iv) a sample comprising nucleated blood cells (such as a sample of buffy coat).

In these embodiments with one or more second subjects, the one or more first subjects may be one subject, such that the samples are from the same first subject. Or, in preferred embodiments with one or more second subjects, the one or more first subjects comprises two or more first subjects.

The one or more second subjects may be one subject, such that the samples are from the same second subject. However, it is preferred that the samples are provided from a plurality of second subjects.

In some embodiments, the one or more second subjects comprises subjects at high risk of developing disease.

As the one or more first subjects have the disease, they are matched according to this characteristic. The one or more first subjects may be further matched by at least one demographic criterion and/or at least one non-demographic criterion.

As the one or more second subjects do not have the disease, they are matched according to this characteristic. The one or more second subjects may be further matched by at least one demographic criterion and/or at least one non-demographic criterion.

The one or more first subjects may be matched with the one or more second subjects by at least one demographic criterion and/or at least one non-demographic criterion. Thus, in some embodiments, the one or more first subjects are further matched amongst themselves by at least one demographic criterion and/or at least one non-demographic criterion, the one or more second subjects are further matched amongst themselves by at least one demographic criterion and/or at least one non-demographic criterion, and the one or more first subjects are matched with the one or more second subjects by at least one demographic criterion and/or at least one non-demographic criterion. In some embodiments, the same demographic criterion and/or non-demographic criterion is used to further match the one or more first subjects amongst themselves, to further match the one or more second subjects amongst themselves, and to match the one or more first subjects with the one or more second subjects.

Methods of preparing the database may be performed with samples from any number of subjects that is adequate to provide the statistical power required in the technical field, for example, to assess subjects as having a disease or as having a disease classification, to a clinically accepted standard.

The number of first subjects and the number of second subjects may be the same, or may be different.

In some embodiments, the one or more first subjects comprises at least about 10 subjects, at least about 20 subjects, at least about 30 subjects, at least about 40 subjects, at least about 50 subjects, at least about 60 subjects, at least about 70 subjects, at least about 80 subjects, at least about 90 subjects, or at least about 100 subjects. Preferably, the one or more first subjects comprises at least about 80 subjects.

In some embodiments, the one or more second subjects comprises at least about 10 subjects, at least about 20 subjects, at least about 30 subjects, at least about 40 subjects, at least about 50 subjects, at least about 60 subjects, at least about 70 subjects, at least about 80 subjects, at least about 90 subjects, or at least about 100 subjects. Preferably, the one or more second subjects comprises at least about 50 subjects.

In some embodiments, the one or more first subjects comprises at least about 80 subjects and the one or more second subjects comprises at least about 50 subjects.

In preferred embodiments, the one or more first subjects and one or more second subjects are human.

In some embodiments, the subjects do not comprise a pregnant woman.

The methods described above involve either a plurality of first subjects with different classifications of the same disease; or one or more first subjects with a disease and one or more second subjects without the disease. Above and herein, it will be apparent to the reader that some attributes of embodiments concerning the one or more first subjects (e.g. the attribute of disease) may equally apply to embodiments concerning the plurality of first subjects. Thus, where applicable, descriptions of the one or more first subjects apply to the plurality of first subjects.

### DNA Extraction and Digestion

In some embodiments, the methods of preparing the database comprise extracting DNA from samples to produce extracted DNA. In other embodiments, the extracted DNA is provided, such that the method itself does not comprise the extraction process.

Genomic DNA can be extracted from samples such as samples of disease tissue, samples of healthy tissue, samples of whole blood, and/or samples comprising nucleated blood cells (such as samples of buffy coat). cfDNA can be extracted from samples such as samples of whole blood and/or samples of plasma.

Extraction may be performed by methods well-known to the skilled person. For tissue samples, a DNA blood mini kit tissue protocol may be used. For whole blood or buffy coat samples, a DNA blood mini kit buffy coat protocol may be used. For plasma samples, a circulating nucleic acids kit may be used. Sonication may be performed on samples comprising genomic DNA.

Methods of preparing the database comprise digesting at least a portion of the DNA using one or more MSREs and/or one or more MDREs. Digestion can be performed, for example at about 37° C., for a long enough period for substantially complete digestion to occur, i.e. further incubation does not lead to any measurable increase in DNA cleavage. MSREs and MDREs are readily available from well-known commercial suppliers, such as ThermoFisher, New England Biolabs, Promega, etc.

MSREs include, but are not limited to: AatII, AccI, AccII, AciI, AclI, AfeI, AgeI, Aor13HI, Aor51HI, AscI, AsiSI, AvaI, BceAI, BmgBI, BsaAI, BsaHI, BsiEI, BsiWI, BsmBI, BspDI, BspT104I, BssHII, BstBI, BstUI, Cfr10I, ClaI, CpoI, DpnII, EagI, Eco52I, FauI, FseI, FspI, HaeII, HapII, HgaI, HhaI, HinP1I, HpaII, Hpy99I, HpyCH4IV, KasI, MluI, MspI, NaeI, NarI, NgoMIV, NotI, NruI, NsbI, PaeR7I, PluTI, PmaCI, PmlI, Psp1406I, PvuI, RsrII, SacII, SalI, ScrFI, SfoI, SgrAI, SmaI, SnaBI, SrfI, TspMI, ZraI.

MDREs include, but are not limited to: BspEI, BtgZI, FspEI, GlaI, LpnPI, McrBC, MspJI, XhoI, XmaI.

Methods of preparing the database may use one or more MSREs only (i.e. no MDREs), one or more MDREs only (i.e. no MSREs), or a mixture of both (i.e. one or more MSREs and one or more MDREs).

In preferred embodiments, methods of preparing the database use one or more MSREs only. Using only MSREs produces digested DNA in which methylated CpG sites are intact but unmethylated CpG sites are cleaved. Thus, for any particular MSRE recognition site in a sample, a higher percentage of methylation at the site leads to a lower extent of cleavage compared to a sample containing a lower percentage of methylation at the site.

In some embodiments, the one or more MSREs comprises (and preferably consists of) HinPII and AciI. This pairing of enzymes covers over 99% of CpG islands in the human genome. HinPII (sometimes known as Hin6I) recognises the sequence GCGC and cleaves after the first G to leave a 2 nucleotide 5′ overhang (5′-G/CGC). AciI recognises the sequence CCGC and cleaves after the first C to leave a 2 nucleotide 5′ overhang (5′-C/CGC).

In some embodiments, the one or more MSREs comprises (and preferably consists of) HinPII, AciI and HpaII.

In some embodiments, the one or more MSREs and/or one or more MDREs comprises (and preferably consists of) HinPII, AciI, HpaII, and HpyCH4IV.

In some embodiments, the MSREs and/or MDREs used with the invention have different recognition sites. Preferred methods do not use restriction enzyme isoschizomers, where one of the enzymes recognizes both the methylated and unmethylated forms of the restriction site while the other recognizes only one of these forms.

The MSREs and/or MDREs may be inactivated after digestion, prior to sequencing. In some embodiments, the inactivation is heat inactivation, for example by heating to 65° C.

In some embodiments, a portion of one or more samples, for example a portion of samples comprising cfDNA, may be reserved as undigested DNA. The undigested DNA may (1) be sequenced as a control, for example to ensure that sites cleaved by the MSREs and/or MDREs in digestion were uncleaved prior to digestion; (2) undergo another procedure such as bisulfite sequencing; or (3) be used to search for non-methylation genomic data (such as mutations) in a multiomics approach. These are further described below.

Preferred methods of preparing the database do not include a step of bisulfite conversion. Other preferred methods include no step in which chemical changes are made to nucleobases within DNA e.g. no bisulfite conversion, no TAPS conversion, etc. TAPS refers to TET-assisted pyridine borane sequencing.

Methods of the invention may be performed according to the disclosure in Israel Patent Application No. 293202 (incorporated by reference in its entirety), for example by using the MSRE/MDRE compositions and digestion methods disclosed therein.

### Sequencing

Digestion is followed by high-throughput sequencing, to identify cfDNA CpG loci and associated methylation levels in samples. Preferably, at least about 100,000, at least about 200,000, at least about 300,000, at least about 400,000, at least about 500,000, at least about 1,000,000, at least about 2,000,000, at least about 3,000,000, at least about 4,000,000, at least about 5,000,000, at least about 6,000,000, at least about 10,000,000, at least about 15,000,000, at least about 20,000,000, at least about 25,000,000, or at least about 28,000,000 cfDNA CpG loci and their methylation levels are identified by the sequencing. Most preferably, at least about 6,000,000 cfDNA CpG loci and their methylation levels are identified by the sequencing.

In some embodiments, the high-throughput sequencing is next-generation sequencing (NGS). Examples of NGS techniques include sequencing-by-synthesis and sequencing-by-ligation (employed, for example, by Illumina Inc., Life Technologies Inc., PacBio, and Roche), nanopore sequencing methods and electronic detection-based methods such as Ion Torrent™ technology (Life Technologies Inc.). NGS may be performed using various sequencing instruments and platforms, including but not limited to: Novaseq™, Nextseq™ and MiSeq™ (Illumina), 454 Sequencing (Roche), Ion Chef™ (ThermoFisher), SOLID® (ThermoFisher) and Sequel II™ (Pacific Biosciences). Appropriate platform-designed sequencing adapters are used for preparing the sequencing library, and are readily available from the platforms' manufacturers.

In some embodiments, the high-throughput sequencing is whole genome (WG) sequencing. In some embodiments, the method comprises target enrichment prior to high-throughput sequencing, such as hybridisation capture or amplicon-based target enrichment. This can be used to sequence specific regions of genomic DNA and cfDNA.

In some embodiments, sequencing comprises converting extracted DNA into a sequencing library, and performing sequencing on the library.

Library preparation for the major high-throughput sequencing platforms involves ligation of specific adapter oligonucleotides, also termed “sequencing adapters”, to the DNA fragments to be sequenced. Sequencing adapters typically include platform-specific sequences for fragment recognition by a particular sequencer e.g. sequences that enable ligated molecules to bind to the flow cells of Illumina platforms (e.g. the P5 and P7 sequences). Each sequencing instrument provider typically sells a specific set of sequences for this purpose.

Sequencing adapters can include sites for binding to a universal set of PCR primers. This permits multiple adapter-ligated DNA molecules to be amplified in parallel by PCR, using a single set of primers.

Sequencing adapters can include sample indices, which are sequences that enable multiple samples to be combined, and then sequenced together (i.e. multiplexed) on the same instrument flow cell or chip. Each sample index, typically 6-10 nucleotides, is specific to a given sample and is used for de-multiplexing during downstream data analysis to assign individual sequence reads to the correct sample. Sequencing adapters may contain single or dual sample indexes depending on the number of libraries combined and the level of accuracy desired.

Sequencing adapters can include unique molecular identifiers (UMIs) to provide molecular tracking, error correction and increased accuracy during sequencing. UMIs are short sequences, typically 5-20 bases in length, used to uniquely identify original molecules in a sample library. As each nucleic acid in the starting material is tagged to provide a unique molecular barcode, bioinformatics software can filter out duplicate reads and PCR errors with a high level of accuracy and report unique reads, removing the identified errors before final data analysis.

In some embodiments, sequencing adapters include both a sample barcode sequence and a UMI.

In some embodiments, sequencing adapters allow for paired-end sequencing.

In some embodiments, methods of the invention use Y-shaped sequencing adapters i.e. adapters consisting of two single-stranded oligonucleotides which anneal to provide a double-stranded stem and two single-stranded ‘arms’. In other embodiments, methods of the invention use hairpin sequencing adapters i.e. a single-stranded oligonucleotide whose 5′ and 3′ termini anneal to provide a double-stranded stem. For both Y-shaped and hairpin adapters the double-stranded stem can include a short single-stranded overhang e.g. a single A or T nucleotide. For both Y-shaped and hairpin adapters the double-stranded stem can be ligated to a cfDNA fragment, to prepare a sequencing library.

Suitable sequencing adapters for use in methods of the invention may thus be TruSeq™ or AmpliSeq™ or TruSight™ adapters (for use on the Illumina platform) or SMRTbell™ adapters (for use on the PacBio platform).

Where sequencing adapters are added by ligation, this usually occurs at both ends of the DNA to be sequenced.

Restriction digestion can leave blunt-ends, but typically produces a single-stranded overhang. Library preparation steps can either preserve this overhang (i.e. add complementary nucleotides) or remove it. As the sequence of a post-digestion terminal single-stranded overhang can include useful information then it is preferred to add sequencing adapters in a way which preserves the overhang e.g. using enzymatic ligation in which a ligase enzyme covalently links a sequencing adapter to a DNA fragment where the terminal sequence of the adapter is complementary to the terminal sequence obtained using the restriction enzyme, or by using a polymerase to add complementary nucleotides and generate a blunt-ended fragment.

In addition to removing or filling in single-strand overhangs, end repair methods carried out before adapter ligation can ensure that DNA molecules contain 5′ phosphate and 3′ hydroxyl groups.

For some libraries, incorporation of a non-templated deoxyadenosine 5′-monophosphate (dAMP) onto the 3′ end of blunted DNA fragments is used in library preparation (a process known as dA-tailing). dA-tails prevent concatemer formation during downstream ligation steps and enable DNA fragments to be ligated to adapter oligonucleotides with complementary dT-overhangs.

Restriction digestion typically takes place in the presence of high levels of Mg⁺⁺. Sequencing library preparation may also rely on Mg⁺⁺, so standard library prep buffers include Mg⁺⁺. In this situation, however, addition of a standard library prep buffer can lead to an excess of Mg⁺⁺ which can inhibit efficiency of downstream steps. Thus added reagents may include a lower level of Mg⁺⁺ than would normally be the case for library preparation.

As an alternative approach to using lower levels of Mg⁺⁺, it is possible to add a chelating agent after digestion, which can remove the need for removal or dilution of excess Mg⁺⁺ for downstream amplification step(s). The addition of a chelating agent at the concentrations disclosed herein does not impair subsequent sequencing. The chelating agent can be added to provide an amplification reaction mix comprising the chelating agent and a divalent cation at a molar ratio of between 1:20 to 2:1. For instance, the reaction mix may include 8-20 mM Mg++ e.g. about 10 mM magnesium. For instance, amplification may be carried out in a reaction mix comprising between 3-4 mM chelating agent and 4 mM Mg⁺⁺. The chelating agent may comprise one or both of EDTA and EGTA.

After library preparation, the prepared DNA molecules can be sequenced, to provide a plurality of “sequence reads”. These sequence reads are then subjected to data processing e.g. to remove sequences which do not fulfil desired quality criteria, to remove duplicates, to correct sequencing errors, to map sequences onto a reference genome, to count the number of sequence reads, etc. Computer software is readily available for performing these steps.

Sequence reads can be mapped to a reference genome i.e. a previously identified genome sequence, whether partial or complete, assembled as a representative example of a species or subject. A reference genome is typically haploid, and typically does not represent the genome of a single individual of the species but rather is a mosaic of the genomes of several individuals. A reference genome for the methods of the present invention is typically a human reference genome e.g. a complete human genome, such as the human genome assemblies available at the website of the National Center for Biotechnology Information or at the University of California, Santa Cruz, Genome Browser. An example of a suitable reference genome for human studies is the ‘hg18’ genome assembly. As an alternative, the more recent GRCh38 major assembly can be used (up to patch p13).

Mapping aligns sequence reads to the reference genome, to identify the location of the reads within the reference genome. The sequence reads that align are designated as being “mapped”. The alignment process aims to maximize the possibility for obtaining regions of sequence identity across the various sequences in the alignment, allowing mismatches, indels and/or clipping of some short fragments on the two ends of the reads. The number of sequence reads mapped to a certain genomic locus is referred to as the “read count” or “copy number” of this genomic locus. It is not necessary to map all sequence reads which are obtained; indeed, it is not unusual that a portion of sequence reads obtained in any given experiment will not be mappable.

Any particular cfDNA CpG locus can feature in multiple sequence reads, which can be sequence reads derived from the same original cfDNA molecule and/or from different cfDNA molecules which span the locus. In some embodiments, sequencing is performed at an average depth of at least about 10×, at least about 80×, at least about 100×, at least about 200×, at least about 300×, at least about 400×, at least about 500×, at least about 600×, or at least about 700×. Preferably, sequencing is performed at an average depth of at least 165×, at least about 225×, or at least about 600×. Most preferred is sequencing performed at an average depth of at least about 600×.

In some embodiments, sequencing of samples comprising cfDNA, and particularly samples of plasma, is performed at a greater average depth than sequencing of other types of sample. In some embodiments, samples comprising cfDNA are sequenced at a higher average depth, such as an average depth of at least about 600×, compared to other types of sample that are sequenced to lower average depth, such as an average depth of at least about 10×, at least about 80×, or at least about 100×. Methods of the invention detect more methylation sites than BS at increased sequencing depths, for example because MSREs and MDREs used in methods of the invention are less noisy than bisulfite sequencing (which may convert residues inappropriately).

Methods disclosed herein do not require differential adapter tagging of methylated vs. unmethylated DNA molecules. The same population of adapters can be used for all molecules.

### Determining Methylation Levels

Methylation levels are determined for cfDNA CpG loci identified by sequencing (see above). Methylation levels for a given locus are determined in each of the samples from each of the subjects, such that a methylation level is separately determined for samples (i), (ii), (iii), (iv) (v) and/or (vi).

Methylation levels can be determined according to how often certain restriction enzymes do (or do not) cleave at their recognition site during digestion. For example, where digestion used an MSRE, sequence reads which span a particular, “target”, locus are derived from molecules which were not cleaved i.e. which (with complete digestion) were methylated at the target locus. How often a recognition site is (or is not) cleaved may be expressed as Hitspan 100.

Methylation levels may be determined relative to a “reference” locus. For example, the methylation level of a target locus can be calculated by dividing its read count by an expected read count of the target locus (e.g. the read count which would be expected if the target locus was fully methylated, and thus uncleaved by an MSRE). The expected read count may be determined using, for instance: (i) the read count of a reference locus that is not cut by the restriction enzyme; (ii) the average read count of a plurality of such reference loci; or (iii) the read count of a reference locus in an undigested reference sample (e.g. a portion of the extracted DNA reserved as undigested DNA), optionally corrected for sequencing depth differences. For example, methylation level may be inferred by comparing the Hitspan100 in a digested sample compared to a reference locus in an undigested sample.

The reference locus may be a different locus compared to the target locus. For example, the reference locus and the target locus may be present in the samples from the one or more first subjects. The reference locus may be in a sample other than those from the one or more first subjects and one or more second subjects, such as an artificial sample comprising a locus with a known methylation level.

Alternatively, the reference locus may be the same locus as the target locus, with the reference locus and target locus in different samples. For example, the target locus may be present in samples from the one or more first subjects, and the reference locus may be in samples from the one or more second subjects. The target locus may be present in samples from one of a plurality of first subjects, and the reference locus may be the same locus in samples from a another one of the plurality of first subjects-for example, in first subjects that have a different disease classifications.

Methylation level may also be determined without use of a reference locus. For example, the expected read count for a target locus may be determined as the sum of the read count at the target locus (indicating methylation, where an MSRE is used) plus the sum of the read counts whose termini map to the target locus (indicating non-methylation), taking account where necessary of any end-repair which took place during library preparation. Therefore, a methylation level may be determined without reference to other loci or other samples, based on the ‘raw’ or ‘absolute’ level of methylation at the target locus. Methylation levels determined without use of a reference locus are also known as “methylation signals”.

These calculations can thus provide, for any given cfDNA CpG locus, the proportion of cfDNA molecules in a sample which were methylated at that locus. Conversely, similar calculations can provide the proportion of a particular cfDNA CpG locus which were unmethylated. These figures can be expressed as a percentage, a fraction, a normalised value, etc.

### Controls

Methods of preparing the database may use positive and/or negative controls to determine that the methods have been performed correctly, or to normalise methylation levels that have been determined. In this way, “controls” perform a different function to the “reference loci” above: reference loci are used to determine a methylation level, whereas controls are used to verify the performance of experimental methods or normalise methylation levels that have been determined.

In some embodiments, parallel digestion and sequencing can be performed as a control on one or more of:
A DNA control which does not contain a recognition site for the restriction enzymes used for digestion. If this DNA is cleaved, this indicates that the method has not performed correctly.
A DNA control (optionally containing a recognition site for the restriction enzymes) which does not undergo digestion. If this undigested DNA is cleaved, this indicates that the method has not performed correctly.
A DNA control which contains a fully methylated recognition site for the restriction enzymes used for digestion. If this DNA is cleaved when a method uses only MSREs, this indicates that the method has not performed correctly (and conversely for MDREs).
A DNA control which contains a fully unmethylated recognition site for the restriction enzymes used for digestion. If this DNA is not fully cleaved when a method uses only MSREs, this indicates that the method has not performed correctly (and conversely for MDREs).

These DNA controls can also be used to normalise methylation levels, as a reference point for analysis, for checking completeness of digestion, etc. As mentioned above, for instance, if fragments are obtained using MSRE digestion then it can be useful in a downstream NGS experiment to know the expected read count, and one way of obtaining this value is to look at the read count for DNA which does not contain the recognition site for the MSRE, or at the read count for DNA which contains the recognition site but is fully methylated.

For these purposes, it is preferred that the DNA control should be similar in size and composition to cfDNA molecules which contain CpG sites of interest. Thus, although it is possible to use synthetic DNA or PCR amplicons or bacterial plasmid DNA as an unmethylated control, these are more useful if they have sizes which are similar to cfDNA (e.g. a long synthetic DNA, or an appropriately-sized restriction fragment prepared from a plasmid).

Control experiments can be performed internally in a sample, or externally. For an internal control, control DNA can be present in a sample already (e.g. cfDNA containing a CpG site which is known to be ubiquitously (un)methylated, or cfDNA which does not contain a recognition site for the restriction enzymes being used) and/or can be added (e.g. synthetic DNA). The control DNA can therefore be processed in combination with the cfDNA, and experiences the same conditions as the cfDNA, and so a method can involve co-amplification of a restriction cfDNA locus and a control locus. For an external control, control DNA is subjected to the same treatment as the cfDNA but not as part of the same reaction mixture.

Thus control DNA, like cfDNA, can be digested with restriction enzymes and then subjected to sequencing.

### Preparing the Database

After sequencing, the database is prepared by recording (at least) cfDNA CpG loci, the methylation levels determined for each cfDNA CpG locus in the samples, and a sample identifier and subject identifier for determined methylation levels.

A sample identifier allows a methylation level for a cfDNA CpG locus to be associated with the sample (or type of sample) from which the DNA containing the locus was extracted. The sample identifier will usually correspond to a specific sample (i.e. each sample from each subject receives its own sample identifier), but may sometimes correspond to a general type of sample (e.g. each sample of disease tissue receives the same sample identifier regardless of which subjects the samples were provided from). The sample identifier can be any means of associating a methylation level with the sample from which it was determined, such as a number or barcode that (1) is recorded in the database and (2) can be associated with a sample using a key in a separate log.

A subject identifier allows a sample to be associated with the subject (or type of subject) from which it was derived. The subject identifier may correspond to a specific subject (i.e. each subject receives their own sample identifier), or more generally to a type of subject (e.g. each of the one or more first subjects with a disease receives the same subject identifier, or optionally, each of the one or more first subjects with the same disease classification receives the same subject identifier). The subject identifier can be any means of associating a sample with the subject from whom it was derived, such as a number or barcode that (1) is recorded in the database and (2) can be associated with a subject using a key in a separate log.

The sample identifier and subject identifier may be used together to associate a particular methylation level with a particular sample and a particular subject. The sample identifier and subject identifier may be the same identifier (e.g. the same barcode) if the identifier allows association both of a methylation level with a sample (or type of sample) and of a sample with a subject (or type of subject).

The database may be any record comprising information identifying the cfDNA CpG loci, methylation level, the sample identifier, and the subject identifier. For example, the database may be a table containing rows of loci, and the methylation level of the loci in columns, organised by sample identifier and subject identifier.

In some embodiments, the database may also comprise any other information useful for selecting markers of disease, such as the disease classification of a subject from whom a sample was derived (such as the stage of the disease of the subject; the subtype of disease; the type of diseased tissue; the severity of disease; the prognosis of the subject); the risk of the subject developing a disease; demographic criteria for the subject; copy number analysis; and/or nucleosomal positioning integrity analysis.

In some embodiments, the database comprises the results of copy number analysis and/or the results of nucleosomal positioning integrity analysis. Methods of the invention lead to improved copy number integrity (for example, copy number integrity achieved by methods of the invention and copy number integrity achieved in untreated control plasma exhibit a Pearson correlation of at least about 0.6, at least about 0.7, at least about 0.8, or at least about 0.9). Methods of the invention lead to improved nucleosomal positioning integrity (for example, nucleosomal positioning integrity achieved by methods of the invention and nucleosomal positioning integrity achieved in untreated control plasma exhibit a Pearson correlation of at least about 0.6, at least about 0.7, at least about 0.8, or at least about 0.9). In some embodiments, methods of the invention have an average sequencing noise per nucleotide of less than about 100×10⁻⁷, or less than about 60×10⁻⁷, or less than about 50×10⁻⁷.

Preparing the database may comprise recording further information. Preparing the database may comprise recording at least 1 b (base) of nucleotide sequence at the 5′ ends of the cfDNA CpG loci, for example recording at least 10 b, 15 b, 20 b, 25 b, 50 b, 100 b, 150 b, 200 b, 250 b, 500 b, 1,000 b, 1,500 b, or 2,000 b adjacent to the 5′ ends of the cfDNA CpG loci. Preparing the database may comprise recording at least 1 b of nucleotide sequence at the 3′ ends of the cfDNA CpG loci, for example recording at least 10 b, 15 b, 20 b, 25 b, 50 b, 100 b, 150 b, 200 b, 250 b, 500 b, 1,000 b, 1,500 b, or 2,000 b adjacent to the 3′ ends of the cfDNA CpG loci. Preparing the database may comprise recording at least 1 b of nucleotide sequence at the 5′ ends and 3′ ends of the cfDNA CpG loci, for example recording at least 10 b, 15 b, 20 b, 25 b, 50 b, 100 b, 150 b, 200 b, 250 b, 500 b, 1,000 b, 1,500 b, or 2,000 b adjacent to the 5′ ends of the cfDNA CpG loci and recording at least 10 b, 15 b, 20 b, 25 b, 50 b, 100 b, 150 b, 200 b, 250 b, 500 b, 1,000 b, 1,500 b, or 2,000 b adjacent to the 3′ ends of the cfDNA CpG loci. In particularly preferred embodiments, preparing the database comprises recording at least 100 b of nucleotide sequence adjacent to the 5′ end of the cfDNA CpG loci and recording at least 100 b of nucleotide sequence adjacent to the 3′ end of the cfDNA CpG loci.

The database may further comprise information concerning non-methylation analytes, such as mutations, for informing a multiomics approach (see below).

### Differentially Methylated Markers

In another aspect of the invention, methods are provided for identifying at least one differentially methylated marker using a database prepared according to the methods described above.

Markers of the invention are differentially methylated cfDNA CpG loci. “Differentially methylated” refers to loci that have a different methylation level in samples from subjects with different diagnoses or different disease classifications, such that the presence or absence of the marker indicates that the subject is more likely or less likely to have the disease or the disease classification. For example, differentially methylated loci may have different methylation levels in samples from subjects with a disease compared to samples from subjects without a disease; in samples from subjects with different disease classifications (such as different severity of the disease, different subtypes of the disease, different types of diseased tissue, different disease severities, different amenability to treatment, or different prognoses); or in samples from subjects with a disease, compared to samples from subjects without a disease but at high risk of developing disease.

In some embodiments, markers of the invention may be differentially methylated in samples from subsets of subjects from a plurality of first subjects, wherein subjects across all subsets each have different classifications of the disease, and subjects in individual subsets have the same classification of the disease (e.g. in one or more primary first subjects compared to one or more other first subjects). Classification of disease may be severity, disease subtype, type of diseased tissue, prognosis, and/or amenability to treatment. For example, one or more primary first subjects may have a disease classification and one or more other first subjects may not have the disease classification (and so may have a different disease classification). cfDNA CpG loci that have different methylation levels in samples of the one or more primary first subjects compared to samples of the one or more other first subjects, are differentially methylated markers indicative of the disease classification.

In some embodiments, markers of the invention may be differentially methylated in samples from different subjects, for example the markers may be differentially methylated in the samples from the one or more first subjects compared to the samples from the one or more second subjects, or the markers may be differentially methylated in different subsets of a plurality of one or more first subjects according to different disease classifications. Markers of the invention may be differentially methylated in the samples comprising cfDNA from the one or more first subjects compared to samples comprising cfDNA from the one or more second subjects. In some embodiments, a cfDNA CpG locus may exhibit differential methylation in a sample of disease tissue and a sample comprising cfDNA from the one or more first subjects, versus a sample of healthy tissue from the one or more first subjects and samples from one or more second subjects.

In some embodiments the differential methylation is hypermethylation (e.g. the methylation level of a locus is greater in samples of disease tissue and samples comprising cfDNA from the one or more first subjects, compared to samples of healthy tissue from the one or more first subjects and the samples from the one or more second subjects, and/or compared to samples comprising nucleated blood cells from the one or more first subjects and one or more second subjects). In some embodiments, the differential methylation is hypomethylation (e.g. the methylation level of a locus is lower in samples of disease tissue and samples comprising cfDNA from the one or more first subjects, compared to samples of healthy tissue from the one or more first subjects and samples from the one or more second subjects, and/or compared to samples comprising nucleated blood cells from the one or more first subjects and one or more second subjects).

Differentially methylated markers have statistically significant differences in methylation between subjects. Statistical significance can be quantified e.g. by calculating a p-value. Methods of determining statistical significance, including calculation of p-value, will be well-known to the person skilled in the art, such as performing a Student's t-test. In some embodiments, cfDNA CpG loci may be rank-ordered according to the statistical significance of their difference in methylation levels, and identified as markers if the loci have statistically significant differences (such as a p-value of less than about 0.05, less than about 0.01, less than about 0.005, less than about 0.001, less than about 0.0005, less than about 0.0001, less than about 0.00005, or less than about 0.00001). Alternatively, top-ranking loci may be identified as markers (such as selecting at least about 2 loci with the most significant difference, or selecting at least about 5, at least about 10, at least about 15, at least about 20, at least about 25, at least about 50, at least about 100, at least about 200, at least about 300, at least about 400, at least about 500, at least about 1,000, at least about 2,000, at least about 3,000, at least about 4,000, at least about 5,000, at least about 6,000, at least about 7,000, at least about 8,000, at least about 9,000, or at least about 10,000 loci-most preferably, selecting at least about 1,000 loci). In addition, or alternatively, statistical significance may be determine by machine leaning methods, such as methods that determine a mathematical vector for a locus.

Differentially methylated markers identified by the above methods can be further investigated for their functional significance, for example their roles in biological processes or metabolic pathways. Proximal gene symbols may be extracted for the markers. It is then possible to calculate the enrichment of differentially methylated markers for predefined sets of biological functions, curated pathways and cancer-related genes, for example using gene set enrichment analysis (GSEA) or gene ontology enrichment analysis. GSEA identifies enrichments in transcriptional regulation and developmental control. This step may be performed by machine learning algorithms.

Specific loci may be identified as markers using a feature selection method, such as minimum redundancy maximum relevance (mRMR).

Advantageously, these methods are able to identify markers that cannot be, or have not been, identified using data obtained from TCGA or whole-genome bisulfite sequencing (BS). In BS, treatment with bisulfite converts unmethylated cytosines to uracil, while methylated cytosines remain unchanged. The comparison of methylation levels before and after bisulfite treatment gives an estimate of DNA methylation. However, BS has several drawbacks, including rapid DNA degradation (due to the chemical harshness of the technique), strong bias (in that cytosine conversion is affected by the surrounding sequence), and high noise (in that there may be incomplete conversion of unmethylated cytosines to uracil, and/or erroneous conversion of methylated cytosines to uracil, leading to a high ‘false methylation’ rate). The inventors consider that methods of the invention identify markers not identifiable by BS, in part because of improved mapping rates of sequence reads (for example of at least about 90%) and/or improved unique mapping rates (for example of at least about 90%). Thus, ‘low background’ markers can be identified using methods of the invention which cannot or have not been identified using BS, in part because of the reduced noise of methods of the invention.

### Selecting a Panel of Differentially Methylated Markers

In another aspect, the invention provides methods of selecting a panel of differentially methylated markers that have been identified by the methods described above. The panel of markers is useful for assessing a test subject (e.g. diagnosing the test subject, or determining whether the test subject has a particular disease classification such as disease stage, disease severity, subtype of disease, type of diseased tissue, prognosis), because seldom does a single marker adequately assess a test subject.

Accordingly, the test subjects may be different subjects from the one or more first subjects and one or more second subjects, and may be different from the plurality of first subjects, used in the methods of preparing the database.

Alternatively, the test subjects may be the same as the one or more first subjects and/or one or more second subjects, or plurality of first subjects, used in the methods of preparing the database and identifying the differentially methylated markers. In this way, the database is used as training data for the panel of markers.

Thus, when selecting a panel of markers, the markers may be used to assess subjects the samples of whom were used in methods of preparing the database. For example, a panel of markers may be used to assess whether a test subject would be a first subject or a second subject according to the cfDNA CpG loci and methylation levels recorded in the database, and the performance of the markers can be evaluated according to whether the subject is correctly assessed as a first subject or a second subject. Similarly, a panel of markers may be used to assess whether a test subject would be a primary first subject or an other first subject according to the cfDNA CpG loci and methylation levels recorded in the database, and the performance of the markers can be evaluated according to whether the subject is correctly assessed as a primary first subject or an other first subject. Thus it can be determined how a panel of markers performs in terms of assessing subjects correctly (e.g. as true positives, true negatives) or incorrectly (e.g. as false positives, false negatives), for example according to whether the subject is in reality a first subject or a second subject (or a primary first subject or an other first subject). An ROC curve can be constructed using this information, the area under which (AUC) indicates how accurate the panel of markers is assessing disease.

The panel of markers is selected to assess test subjects with high sensitivity and specificity, for example with an area under an ROC curve (AUC) of at least about 0.8, at least about 0.85, at least about 0.9, at least about 0.93, or at least about 0.95. Preferably the panel of markers assess test subjects with an area under an ROC curve (AUC) of at least about 0.93.

In some embodiments, it is useful to focus on samples comprising a high proportion of DNA originating from disease tissue in order to select a panel of markers most appropriate to assess test subjects. Therefore, somatic mutation analysis may be performed, in tissue samples and samples comprising cfDNA, prior to the methods of identifying markers, to show samples with a high proportion of DNA originating from disease tissue. Methods of identifying at least one differentially methylated marker described above may then be performed on these samples comprising a high proportion of DNA originating from disease tissue.

In some embodiments (for example wherein a PCR assay for detecting the panel of markers is used to assess test subjects), at least about 15 markers, at least about 20 markers, at least about 25 markers, at least about 30 markers, at least about 35 markers, at least about 40 markers, at least about 45 markers, at least about 50 markers, at least about 55 markers, at least about 60 markers, at least about 65 markers, at least about 70 markers, at least about 75 markers, at least about 80 markers, at least about 85 markers, at least about 90 markers, at least about 95 markers, or at least about 100 markers, are selected.

In some embodiments (for example wherein a PCR assay for detecting the panel of markers is used to assess test subjects), less than about 300 markers, less than about 200 markers, less than about 150 markers, less than about 120 markers, less than about 100 markers, less than about 95 markers, less than about 90 markers, less than about 85 markers, less than about 80 markers, less than about 75 markers, less than about 70 markers, less than about 65 markers, less than about 60 markers, less than about 55 markers, less than about 50 markers, less than about 45 markers, less than about 40 markers, less than about 35 markers, less than about 30 markers, less than about 25 markers, less than about 20 markers, or less than about 15 markers are selected.

In some embodiments (for example wherein an NGS assay for detecting the panel of markers is used to assess test subjects), at least about 1,000 markers, at least about 1,100 markers, at least about 1,200 markers, at least about 1,300 markers, at least about 1,400 markers, at least about 1,500 markers, at least about 2,000 markers, at least about 2,500 markers, at least about 3,000 markers, at least about 3,500 markers, at least about 4,000 markers, at least about 4,500 markers, at least about 5,000 markers, at least about 6,000 markers, at least about 7,000 markers, at least about 8,000 markers, at least about 9,000 markers, or at least about 10,000 markers, are selected.

In some embodiments, the markers are selected to be synergistic, meaning that markers are selected which when combined correctly assess more subjects (e.g. have increased specificity and/or sensitivity) than the sum of correct assessments made for a marker individually. In some embodiments, markers are identified which are associated with disease, and markers are identified which are associated with the absence of disease.

### Multiomics

In some embodiments, at the same time as determining methylation levels for recording in the database, methods of preparing the database may comprise additional steps to measure and record one or more non-methylation analytes in the samples as potential markers of disease. This is termed a ‘multiomics’ approach. The non-methylation analytes may comprise one or more of a cfDNA non-CpG locus, a mutation, copy number data, nucleosomal positioning data, fragmentomics data, proteomics data, gene expression data, clinical data such as disease symptoms or severity, and metabolite data such as metabolite concentration.

Some non-methylation analytes, such as cfDNA non-CpG loci, may be detectable in cfDNA, for example using methods of preparing the database as described above. cfDNA non-CpG loci comprise loci which are detectable in cfDNA and do not contain a CpG site (or the cytosine residue of a CpG site). For example, a cfDNA non-CpG locus may comprise a wild-type sequence relative to a reference genome (i.e. a sequence not containing mutations). The cfDNA non-CpG locus may nevertheless be a marker of disease (even in the absence of a mutation) by virtue of being detectable in samples comprising cfDNA of subjects with a disease (or with a disease classification) and not being detectable in samples comprising cfDNA of subjects without the disease (or without the disease classification).

The non-methylation analytes may be measured during the aforementioned steps of digestion and sequencing. For example, mutations and/or cfDNA non-CpG loci can be measured during sequencing of digested DNA, and/or during sequencing of a reserved portion of undigested DNA, which can be performed as part of the methods of preparing the database.

Non-methylation analytes may also be measured using other, standard procedures known in the art, for example by measuring known non-methylation analytes (such as metabolite concentrations) in samples. Measurement of known non-methylation analytes may be performed on one or more of the same samples used in methods of the invention, such as samples of plasma and/or samples of tissue. Any of this information may be measured and recorded in methods of the invention for preparing a database comprising cfDNA CpG loci.

In some embodiments, the non-methylation analytes comprise one or more mutations compared to a reference genome, for example one mutation, for example a point mutation, deletion, insertion, or transposition. In some embodiments, the reference genome is determined from a sample of healthy tissue from the one or more first subjects (or the plurality of first subjects). In some embodiments, the reference genome derived from a sample from the one or more second subjects. In some embodiments, the mutation is a passenger mutation, which does not cause disease and instead arises because of disease and provides no selective advantage to the diseased cell. In some embodiments, the non-methylation marker does not comprise repetitive indel mutations.

In some embodiments, the one or more non-methylated analytes comprise one mutation, and the one mutation is proximal to a differentially methylated marker. In some embodiments, the differentially methylated marker is located within about 1,000 bp, within about 500 bp, within about 200 bp, within about 150 bp, within about 100 bp, or within about 50 bp of the mutation. This arrangement of the differentially methylated marker and the mutation may be described as a multiomic region. Multiomic regions allow for detection of two types of analytes (marker and non-methylation analyte) in a single read, and can enhance the specificity of disease detection, for example tumor detection. Preferably, a multiomic region comprises a differentially methylated marker within about 100 bp of a mutation.

### Diseases

All methods of the invention are applicable to any disease associated with differential methylation of loci. The markers identified by methods of the invention may be used to assess subjects. “Assessing” subjects may include diagnosing the subjects (determining the presence or absence of a disease), classifying the subjects with a particular disease classification, or determining the risk of a subject developing disease in future. Disease classifications may include the stage of disease, disease subtype, type of diseased tissue, prognosis, amenability to treatment, or the severity of disease. For example, the markers may be used to assess whether subjects have early-stage cancer, such as stage I cancer or stage II cancer.

In some embodiments, the disease is chronic obstructive pulmonary disease (COPD). In some embodiments, the disease is cancer, such as lung cancer, adenocarcinoma, squamous cell carcinoma, adenosquamous carcinoma, breast cancer, prostate cancer, liver cancer, colorectal cancer, colon cancer, bladder cancer, head and neck cancer, Wilms tumors, ovarian cancer, esophageal cancer, hepatocellular carcinoma, glioblastoma, thyroid carcinoma, leukaemia, or a bone cancer.

In some embodiments, the disease is lung cancer. The lung cancer may be early-stage lung cancer, including stage I lung cancer and/or stage II lung cancer. Disease staging of cancer may be performed by any suitable method, for example according to the American Joint Committee on Cancer staging manual (AJCC)7 and/or AJCC8. In some embodiments, the panel of markers assess whether a subject has a subtype of lung cancer or a stage of lung cancer. In some embodiments, markers selected according to methods of the invention allow diagnosis of lung cancer at an early stage.

In some embodiments, the cancer is invasive. In some embodiments, the cancer is non-invasive.

The markers may allow detection of tumors, including small, low shedding tumors, with high sensitivity and/or high specificity.

In some embodiments, the markers are associated with disease samples or non-disease samples. In some embodiments, the markers are associated with cancer samples or non-cancer samples. In some embodiments, the markers are associated with early-stage cancer samples or later-stage cancer samples. In some embodiments, the markers are associated with subjects with cancer or subjects without cancer but at high risk of developing cancer.

In some embodiments, the markers are determinative of cancer prognosis, for example the markers classify cancer subjects with an average survival time from diagnosis of less than about 12 months, or classify cancer subjects with an average survival time from diagnosis of about 12 months or more. In some embodiments, the markers classify cancer samples based on clinical parameters. The clinical parameters may be one or more of cancer stage, extent of metastasis (M stage), tumor size (T stage), tumor location, or N stage.

In some embodiments, the one or more first subjects (or the one or more primary first subjects) are smokers, for example, they have smoked within 1 year prior to samples being provided. In some embodiments, they have been smokers but are no longer smokers. In some embodiments, they are not smokers and have never been smokers.

In some embodiments, the one or more first subjects (or the one or more primary first subjects), or the one or more second subjects are at high risk of developing lung cancer. Preferably, the one or more second subjects do not have lung cancer but are at high risk of developing lung cancer. High risk may be classified according to the methodology of the US Preventative Services Taskforce (USPSTF), which defines high risk individuals as 55 through 80 years old, are either current smokers or who have quit within the past 15 years, and have a smoking history of 30 pack years or more. In some embodiments, the subject with the disease has a smoking history of about 40 pack years or more. In some embodiments, the subject with the disease is at least about 50 years old, at least about 55 years old, at least about 60 years old, or at least about 65 years old.

In some embodiments, the disease is an immune disorder, such as rheumatoid arthritis and multiple sclerosis. In some embodiments, the disease is a metabolic disorder, such as hyperglycemia, diabetes, and hyperlipidaemia. In some embodiments, the disease is a neurological disorder, such as autism spectrum disorder, Rett syndrome, schizophrenia, Parkinson's disease, Huntington's disease, or Alzheimer's disease.

In some embodiments, the one or more first subjects (or the one or more primary first subjects) may be treatment-naïve at the time samples are provided.

### Systems and Machine Learning Methods

The invention also provides various systems and machine learning methods.

A system can comprise computer processor(s) for performing and/or controlling methods of the invention, and/or for processing the results e.g. for performing calculations based on the results. Methods which are at least partially computer-implemented are provided.

The system may comprise: components for carrying out methods of the invention on at least one cfDNA CpG locus (e.g. at least one CpG site); and computer software stored on a non-transitory computer readable medium, the computer software being able to direct a computer processor to determine a methylation level for the at least one cfDNA CpG locus based on methods of the invention. The computer software may receive data from high-throughput sequencing.

Computer software includes processor-executable instructions that are stored on a non-transitory computer readable medium. The computer software may also include stored data. The computer readable medium is a tangible computer readable medium, such as a compact disc (CD), magnetic storage, optical storage, random access memory (RAM), read only memory (ROM), or any other tangible medium.

Computer-related methods and steps described herein are implemented using software stored on non-volatile or non-transitory computer readable instructions that when executed configure or direct a computer processor or computer to perform the instructions.

Each of the system, server, computing device, and computer described herein can be implemented on one or more computer systems and be configured to communicate over a network. They all may also be implemented on one single computer system. In one embodiment, the computer system includes a bus or other communication mechanism for communicating information, and a hardware processor coupled with bus for processing information.

A computer system also includes a main memory, such as a random-access memory (RAM) or other dynamic storage device, coupled to bus for storing information and instructions to be executed by processor. Main memory also may be used for storing temporary variables or other intermediate information during execution of instructions to be executed by processor. Such instructions, when stored in non-transitory storage media accessible to processor, render computer system into a special-purpose machine that is customized to perform the operations specified in the instructions.

A computer system can include read only memory (ROM) or other static storage device coupled to bus for storing static information and instructions for processor. A storage device, such as a magnetic disk or optical disk, is provided and coupled to bus for storing information and instructions.

A computer system may be coupled via bus to a display, for displaying information to a computer user.

An input device, including alphanumeric and other keys, can be coupled to bus for communicating information and command selections to processor. Another type of user input device is cursor control, such as a mouse, a trackball, or cursor direction keys for communicating direction information and command selections to processor and for controlling cursor movement on display.

Methods of the invention may be performed by a computer system in response to the processor executing one or more sequences of one or more instructions contained in main memory. Such instructions may be read into main memory from another storage medium, such as storage device. Execution of the sequences of instructions contained in main memory causes the processor to perform the process steps described herein. In alternative embodiments, hard-wired circuitry may be used in place of or in combination with software instructions.

Suitable storage media include any non-transitory media that store data and/or instructions that cause a machine to operation in a specific fashion. Common forms of storage media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge.

Storage media are distinct from, but may be used in conjunction with, transmission media. Transmission media participates in transferring information between storage media. For example, transmission media includes coaxial cables, copper wire and fiber optics, including the wires that comprise bus.

A machine learning method may be developed using a panel of markers selected according to methods of the invention, to assess samples according to disease (e.g. to distinguish between disease samples and non-disease samples). In some embodiments, the machine-learning method comprises a machine learning logistic regression model. In some embodiments, the machine-learning method is trained on, or used to analyse, data obtained from subject samples, such as subject samples subject to the digestion and sequencing described herein.

### EXAMPLES

### Example 1

### Development of a Genome-Wide Multiomic Atlas of Early-Stage Lung Cancer Enables Identification of Novel Methylation Markers for Disease Detection

We constructed a novel multiomic atlas through whole genome sequencing (WGS) to map all genetic and epigenetic changes associated with early-stage lung cancer as a tool to identify disease markers.

Samples from 48 early-stage lung cancer subjects (58% stage I) and 29 cancer-free control subjects, all high-risk by US Preventative Services Taskforce (USPSTF), were acquired from academic and commercial biobanks. Tissue (tumor and normal lung), whole blood (WB) and plasma samples were acquired for the lung cancer cases. WB and plasma samples were acquired for the control cases.

Each sample underwent digestion with methylation-sensitive restriction enzymes followed by standard library preparation and sequencing (herein denoted “ECS”). Sequencing of cfDNA was performed at an average depth of 600×; tissue and WB were sequenced at 80×. A subset also underwent WG bisulfite sequencing (BS) and standard WGS for comparison. Data analysis was performed using customized software.

Mapping rate was 99.6%, 99.7% and 85.7% and unique mapping rate was 94.1%, 94.3% and 81.4% for WGS, ECS, and BS samples, respectively. Copy number integrity showed Pearson correlations of 0.9 for ECS and 0.67 for BS. Somatic mutation analysis identified a subset of cases with relatively high ctDNA shedding that were associated with larger tumors, older age and squamous cell carcinoma histology. This subset was further used to identify tumor-derived plasma-based markers and assess fragmentation with high confidence. Shorter ctDNA fragments were observed in some, but not all cases. Additionally, host derived blood-based markers were developed using WG methylation data and copy number data of WB and plasma samples. Results showed these markers enable detection of small, low shedding tumors. A panel of 87 markers was assembled, with 100% sensitivity and 97% specificity to discriminate cancers vs controls. Only 11% of the markers are represented in TCGA data.

ECS generates high integrity sequencing data superior to BS, enabling genome-wide multiomic analyses (methylation, mutation, copy number, nucleosomal positioning, fragmentomics), with methylation signatures beyond the scope of TCGA. The results underscore the potential of ECS as a development platform of novel liquid biopsy markers for early-stage lung cancer detection.

### Example 2

### Machine-Learning-Based Epigenetic Detection of Early-Stage Lung Cancers Using the EpiCheck Liquid Biopsy Platform

The EpiCheck platform combines methylation-sensitive restriction endonuclease (MSRE) digestion with whole genome sequencing to comprehensively map genome-wide DNA methylation changes with high fidelity. In this proof-of-concept study, MSRE-WGS was used to interrogate a liquid biopsy atlas focused on early-stage lung cancers to i) identify informative markers in plasma, and ii) develop a machine learning method to discriminate between cancer and high-risk, non-cancer controls.

The EpiCheck lung cancer atlas was constructed using samples from academic and commercial sources. Plasma samples were collected from 81 cancer subjects and 54 high-risk individuals without cancer. Extracted DNA was digested with methylation-sensitive endonucleases and sequenced at an average depth of 600×. Methylation levels of ~6 million genomic loci were rank ordered using Student's t-test. Gene set enrichment analysis (GSEA) was performed on the top-ranking 1,000 differentially methylated loci. A logistic regression classifier with Lasso regularization was trained on 100,000 loci, and performance was examined by mean AUC using 5-fold cross-validation.

A total of 1,527 loci exhibited significant differential methylation patterns between cancers and controls (p<0.01, FDR corrected). Of these, 876 and 651 lung cancer loci were hypermethylated and hypomethylated, respectively. Biological characterization using GSEA identified enrichments in transcriptional regulation and developmental control. In particular, loci were enriched for Polycomb Repressive Complex regulated genes, suggesting a possible connection to abnormal epigenetic regulation via histone modification in lung cancer. Construction of a machine learning logistic regression model based on the five training folds utilized 252 loci on average, and achieved a mean cross-validation AUC of 0.93 when distinguishing plasma cancer cases vs controls.

Our findings demonstrate that the MSRE-NGS EpiCheck platform identified markers within the plasma methylome for detecting early-stage lung cancer. A machine learning model trained on methylation targets performed with high accuracy in discriminating lung cancer subjects from high-risk healthy individuals.

It will be understood that the inventors' work has been described above by way of example only and modifications may be made while remaining within the scope and spirit of the invention.

### REFERENCES

All references described above are incorporated by reference in their entirety.
Cancer Genome Atlas Research Network et al., Nat Genet. 2013; 45(10): 1113-1120
Gardiner-Garden & Frommer (1987) J Mol Biol 196:261-82
Alcaide et al. (2020) Scientific Reports 10, article 12564
Israel Patent Application No. 293202

1. A method of preparing a database comprising cfDNA CpG loci, the method comprising:
(a) Providing DNA extracted from:
(i) a sample of disease tissue from one or more first subjects with a disease;
(ii) a sample comprising cfDNA from the one or more first subjects; and
(iii) a sample comprising cfDNA from one or more second subjects without the disease,
(b) Digesting at least a portion of the DNA with one or more methylation-sensitive restriction enzymes (MSREs) and/or one or more methylation-dependent restriction enzymes (MDREs), producing digested DNA;
(c) Sequencing the digested DNA with high-throughput sequencing, thereby identifying cfDNA CpG loci;
(d) Determining methylation levels of the cfDNA CpG loci in the samples of (i), the samples of (ii), and the samples of (iii); and
(e) Preparing the database by recording:
the cfDNA CpG loci identified by the sequencing in (c);
the methylation levels determined in (d) for the cfDNA CpG loci;
a sample identifier; and
a subject identifier.

2. The method of claim 1, wherein the one or more second subjects is/are at high risk of developing the disease.

3. The method of claim 1 claims, wherein step (a) comprises providing DNA extracted from:
(iv) a sample comprising nucleated blood cells from the one or more second subjects, and performing steps (b)-(e) also for the DNA extracted from samples of (iv).

4. A method of preparing a database comprising cfDNA CpG loci, the method comprising:
(a) providing DNA extracted from:
(i) a sample of disease tissue from a plurality of first subjects with a disease; and
(ii) a sample comprising cfDNA from the plurality of first subjects,
(b) digesting at least a portion of the DNA with one or more methylation-sensitive restriction enzymes (MSREs) and/or one or more methylation-dependent restriction enzymes (MDREs), producing digested DNA;
(c) sequencing the digested DNA with high-throughput sequencing, thereby identifying cfDNA CpG loci;
(d) determining methylation levels of the cfDNA CpG loci in the samples of (i) and the samples of (ii); and
(e) preparing the database by recording:
the cfDNA CpG loci identified by the sequencing in (c);
the methylation levels determined in (d) for the cfDNA CpG loci;—a sample identifier; and
a subject identifier,
wherein the plurality of first subjects comprises one or more primary first subjects with a disease classification, and one or more other first subjects without the disease classification.

5. The method of claim 4, wherein the disease classification comprises disease stage, disease subtype, disease tissue, disease severity, prognosis, or amenability to treatment.

6. The method of claim 1, wherein step (a) comprises providing DNA extracted from:
(v) a sample of healthy tissue from the one or more first subjects or the plurality of first subjects, and performing steps (b)-(e) also for the DNA extracted from samples of (v).

7. The method of claim 1, wherein step (a) comprises providing DNA extracted from:
(vi) a sample comprising nucleated blood cells from the one or more first subjects or the plurality of first subjects, and performing steps (b)-(e) also for the DNA extracted from samples of (vi).

8. The method of claim 1, comprising reserving prior to step (b) a portion of the DNA as undigested DNA, and performing steps (c)-(d) on the undigested DNA.

9. The method of claim 1, wherein the digested DNA and/or the undigested DNA are subject to target enrichment prior to the sequencing.

10. The method of claim 9, wherein the target enrichment comprises hybridisation capture or amplicon-based target enrichment.

11. The method of claim 1, wherein the samples comprising cfDNA are samples of plasma.

12. The method of claim 3, wherein the samples comprising nucleated blood cells are samples of buffy coat.

13. The method of claim 3, wherein samples of whole blood are provided from the one or more first subjects or the plurality of first subjects, and separated into the samples comprising cfDNA and the samples comprising nucleated blood cells;

14. (canceled)

15. The method of claim 1, wherein one or more first subjects are matched with the one or more second subjects by one or more demographic criteria, wherein the one or more demographic criteria optionally comprise age, race, sex, and/or ethnicity; and/or the one or more first subjects are matched with the one or more second subjects by one or more non-demographic criteria, wherein the one or more non-demographic criteria optionally comprise health status, medical history, disability status, body weight, BMI, smoking status, chronic obstructive pulmonary disease (COPD) status, tumor presence, tumor size, cancer stage, cancer metastasis, and/or psychiatric diagnosis.

16. The method of claim 4, wherein the one or more primary first subjects are matched with the one or more other first subjects by one or more demographic criteria, wherein the one or more demographic criteria optionally comprise age, race, sex, and/or ethnicity; and/or the one or more primary first subjects are matched with the one or more other first subjects by one or more non-demographic criteria, wherein the one or more non-demographic criteria optionally comprise health status, medical history, disability status, body weight, BMI, smoking status, chronic obstructive pulmonary disease (COPD) status, tumor presence, tumor size, cancer stage, cancer metastasis, and/or psychiatric diagnosis.

17-20. (canceled)

21. The method of claim 1, wherein the one or more first subjects or the plurality of first subjects comprises at least about 10, at least about 20, at least about 30, at least about 40, at least about 50, at least about 60, at least about 70, at least about 80, at least about 90, or at least about 100 subjects; and the one or more second subjects comprises at least about 10, at least about 20, at least about 30, at least about 40, at least about 50, at least about 60, at least about 70, at least about 80, at least about 90, or at least about 100 subjects.

22. (canceled)

23. The method of claim 1, wherein preparing the database comprises recording at least 1 b of nucleotide sequence adjacent to the 5′ ends and/or 3′ ends of the cfDNA CpG loci, for example recording at least 10 b, 15 b, 20 b, 25 b, 50 b, 100 b, 150 b, 200 b, 250 b, 500 b, 1,000 b, 1,500 b, or 2,000 b adjacent to the 5′ ends and/or 3′ ends of the cfDNA CpG loci.

24. The method of claim 1, wherein step (e) comprises recording in the database one or more non-methylation analytes measured in the samples, wherein the one or more non-methylation analytes optionally comprise one or more of a cfDNA non-CpG locus, a mutation, copy number data, nucleosomal positioning data. fragmentomics data, proteomics data, gene expression data, clinical data, and metabolite data, and wherein the cfDNA non-CpG locus and/or the mutation, if measured, is/are determined during sequencing of digested DNA and/or during sequencing of a reserved portion of undigested DNA.

25. (canceled)

26. (canceled)

27. A method for identifying at least one differentially methylated marker of disease, the method comprising:
(A) Providing a database comprising cfDNA CpG loci prepared according to claim 1;
(B) Querying the database for cfDNA CpG loci having different methylation levels between the one or more first subjects and the one or more second subjects;
(C) Ranking the cfDNA CpG loci according to statistical significance of the difference in methylation levels; and
(D) Identifying as markers, cfDNA CpG loci having a statistically significant difference in methylation levels.

28. A method for identifying at least one differentially methylated marker of disease, the method comprising:
(A) Providing a database comprising cfDNA CpG loci prepared according to claim 4;
(B) Querying the database for cfDNA CpG loci having different methylation levels between the one or more primary first subjects and the one or more other first subjects;
(C) Ranking the cfDNA CpG loci according to statistical significance of the difference in methylation levels; and
(D) Identifying as markers, cfDNA CpG loci having a statistically significant difference in methylation levels.

29. (canceled)

30. A method of selecting a panel of differentially methylated markers of disease, the method comprising:
(I) Providing differentially methylated markers of disease identified according to claim 27 the and
(II) Selecting at least about 10 of the differentially methylated markers, in order to obtain an area under a ROC curve (AUC) of at least about 0.8 when the markers are used to assess test subjects.

31. The method of claim 30, wherein the selecting comprises selecting at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 of the differentially methylated markers.

32. (canceled)

33. The method of claim 31, further comprising providing means for detecting the selected differentially methylated markers in a sample comprising cfDNA.

34. The method of claim 33, further comprising performing an in vitro method for assessing disease in a test subject comprising detecting the panel of markers of claim 33.

35. The method of claim 1, wherein the disease is chronic obstructive pulmonary disease (COPD) or cancer, optionally wherein the disease is lung cancer, adenocarcinoma, squamous cell carcinoma, adenosquamous carcinoma, breast cancer, prostate cancer, liver cancer, colorectal cancer, colon cancer, bladder cancer, head and neck cancer, Wilms tumors, ovarian cancer, esophageal cancer, hepatocellular carcinoma, glioblastoma, thyroid carcinoma, leukaemia, or a bone cancer.
